(19) United States
(12) Patent Application Publication
(10) Pub. No.: US 2024/0252169 A1
(43) Pub. Date: Aug. 1, 2024

(54) ARTICULATION ASSEMBLY FOR A SURGICAL DEVICE

(71) Applicant: Covidien LP, Mansfield, MA (US)

(72) Inventor: Lan Chang, Shanghai (CN)

(21) Appl. No.: 18/562,392

(22) PCT Filed: May 21, 2021
(86) PCT No.: PCT/CN2021/095023

§ 371 Date: Nov. 20, 2023

(51) Int. Cl. A61B 17/072; A61B 17/00; A61B 17/29; F16C 11/10

(52) U.S. Cl. CPC A61B 17/07207; F16C 11/10; A61B 2017/00367; A61B 2017/07257; A61B 2017/07271; A61B 2017/2927

(57) ABSTRACT

A surgical device includes an elongate body, a tool assembly supported on the elongate body for articulation, and an articulating mechanism for selectively articulating the tool assembly in relation to the elongate body. The articulation mechanism includes an articulation lever and a pair of locking assemblies. The articulation lever is movable to articulate the tool assembly in relation to the elongate body about an articulation axis. The locking assemblies are configured to prevent movement of the articulation lever until one of the locking assemblies is moved from a locked position to an unlocked position. The locking assemblies are movable from their locked positions to their unlocked positions prior to movement of the articulation lever to reduce the force required to manipulate the articulation lever and to facilitate articulation in continuous fashion.

### FIELD

This disclosure is generally related to surgical devices for endoscopic use and, more particularly, to surgical devices including articulation mechanisms for articulating tool assemblies.

### BACKGROUND

Various types of surgical devices used to endoscopically treat tissue are known in the art, and are commonly used, for example, for closure of tissue or organs in transection, resection, and anastomoses procedures, for occlusion of organs in thoracic and abdominal procedures, and for electrosurgically fusing or sealing tissue.

During laparoscopic or endoscopic surgical procedures, access to a surgical site is achieved through a small incision or through a narrow cannula inserted through a small entrance wounds in a patient. Because of limited area available to access the surgical site, many endoscopic devices include mechanisms for articulating the tool assembly of the device in relation to a body portion of the device about a pivot axis.

Typically, articulation mechanisms for articulating a tool assembly of a surgical stapling device include a lever that can be manipulated to articulate the tool assembly. To manipulate the lever, the force of a biasing member must be overcome. This increases the forces required to articulate the tool assembly.

A continuing need exists in the art for an articulating mechanism that can be operated with minimal force.

### SUMMARY

This disclosure is directed to a surgical stapling device including a tool assembly, an elongate body, and an articulation mechanism having an articulation lever and a pair of locking assemblies. The articulation lever is movable to articulate the tool assembly in relation to the elongate body about an articulation axis that is transverse to a longitudinal axis of the elongate body. The locking assemblies are configured to prevent movement of the articulation lever until one of the locking assemblies is moved from a locked position to an unlocked position. The locking assemblies are movable from their locked positions to their unlocked positions prior to movement of the articulation lever to reduce the force required to manipulate the articulation lever and to facilitate articulation in continuous fashion.

Aspects of the disclosure are directed to an articulation assembly for use with a surgical device that includes a housing, an articulation lever, a first ratchet, a second ratchet, a drive member, an articulation link, a first locking assembly, and a second locking assembly. The housing defines a circular bore and a cavity and includes a base wall separating the circular bore from the cavity. The base wall defines an opening that communicates the circular bore with the cavity. The articulation lever is rotatably supported in relation to the circular bore. The first ratchet has an annular array of teeth supported within the circular bore. The second ratchet has an annular array of teeth positioned adjacent the first ratchet. The drive member includes a shaft having a first end coupled to the articulation lever and a second end. The shaft extends through the opening in the base wall of the housing. The drive member is rotatable in response to rotation of the articulation lever. The articulation link is coupled to the second end of the drive member such that rotation of the drive member causes longitudinal movement of the articulation link. The first locking assembly is supported on the articulation lever and includes a first locking member that is movable from a first position engaged with the first ratchet to a second position disengaged with the first ratchet. The first locking member is configured to prevent rotation of the articulation lever in a counterclockwise direction when the first locking member is in its first position. The second locking assembly is supported on the articulation lever and includes a second locking member that is movable from a first position engaged with the second ratchet to a second position disengaged with the second ratchet. The second locking member is configured to prevent rotation of the articulation lever in a clockwise direction when the second locking member is in its first position.

In aspects of the disclosure, the first locking assembly includes a first actuator that is supported on the articulation lever, and the second locking assembly includes a second actuator that is supported on the articulation lever.

In some aspects of the disclosure, the first actuator is movable to move the first locking member from its first position to its second position and the second actuator is movable to move the second locking member from its first position to its second position.

In certain aspects of the disclosure, the first locking assembly includes a first slider that is positioned between the first actuator and the first locking member, and the second locking assembly includes a second slider that is positioned between the second actuator and the second locking member.

In aspects of the disclosure, the first actuator is movable from a first position to a second position to move the first slider from a first position to a second position into engagement with the first locking member to move the first locking member from its first to its second position, and the second actuator is movable from a first position to a second position to move the second slider from a first position to a second position into engagement with the second locking member to move the second locking member from its first position to its second position.

In some aspects of the disclosure, the first actuator and the first slider include abutting cam surfaces such that transverse movement of the first actuator causes longitudinal movement of the first slider.

In certain aspects of the disclosure, the second actuator and the second slider include abutting cam surfaces such that transverse movement of the second actuator causes longitudinal movement of the second slider.

In aspects of the disclosure, the articulation assembly includes a biasing member that is positioned to urge the first and second actuators towards their first positions.

In some aspects of the disclosure, the first and second locking members are formed of a resilient material.

In certain aspects of the disclosure, the first locking member includes a first retention portion and a first ratchet finger, and the second locking member includes a second retention portion and a second ratchet finger.

In aspects of the disclosure, the first ratchet finger is engaged with the first ratchet when the first locking member is in its first position and the second ratchet finger is engaged with the second ratchet when the second locking member is in its first position.

In some aspects of the disclosure, the articulation assembly includes a first biasing member positioned to urge the first slider to its first position and a second biasing member positioned to urge the second slider to its first position.

In certain aspects of the disclosure, the second end of the drive member includes a disc-shaped member, and the articulation assembly includes a cam member and a coupling member.

In aspects of the disclosure, the cam member is supported on the disc-shaped member and coupled to the coupling member such that rotational movement of the drive member causes longitudinal movement of the coupling member.

In aspects of the disclosure, the coupling member is coupled to the articulation link such that longitudinal movement of the coupling member causes longitudinal movement of the articulation link.

In aspects of the disclosure, the tool assembly includes an anvil and a cartridge assembly.

Other aspects of the disclosure are directed to a surgical device that includes an elongate body, a tool assembly, and an articulation assembly. The elongate body defines a first longitudinal axis and has a proximal portion and a distal portion. The tool assembly defines a second longitudinal axis and is pivotably supported on the distal portion of the elongate body by a pivot member. The pivot member defines an articulation axis that is transverse to the first longitudinal axis. The tool assembly is pivotable about the articulation axis in relation to the elongate body between an articulated position in which the first and second longitudinal axes are coaxial and articulated positions in which the first and second longitudinal axes are misaligned. The articulation assembly includes a housing, an articulation lever, a first ratchet, a second ratchet, a drive member, an articulation link, a first locking assembly, and a second locking assembly. The housing defines a circular bore and a cavity and includes a base wall separating the circular bore from the cavity. The base wall defines an opening that communicates the circular bore with the cavity. The articulation lever is rotatably supported in relation to the circular bore. The first ratchet has an annular array of teeth supported within the circular bore. The second ratchet has an annular array of teeth supported adjacent the first ratchet. The drive member includes a shaft having a first end coupled to the articulation lever and a second end. The shaft extends through the opening in the base wall of the housing, and the drive member is rotatable in response to rotation of the articulation lever. The articulation link has a proximal portion and a distal portion. The proximal portion is coupled to the second end of the drive member such that rotation of the drive member causes longitudinal movement of the articulation link. The distal portion of the articulation link is coupled to the tool assembly such that longitudinal movement of the articulation link causes the tool assembly to move between the non-articulated and articulated positions. The first locking assembly is supported on the articulation lever and includes a first locking member that is movable from a first position engaged with the first ratchet to a second position disengaged with the first ratchet. The first locking member is configured to prevent rotation of the articulation lever in a counterclockwise direction when the first locking member is in its first position. The second locking assembly is supported on the articulation lever and includes a second locking member that is movable from a first position engaged with the second ratchet to a second position disengaged with the second ratchet. The second locking member is configured to prevent rotation of the articulation lever in a clockwise direction when the second locking member is in its first position.

Other aspects of the disclosure are directed to an articulation assembly for use with a surgical device that includes a housing, an articulation lever, a first ratchet, a second ratchet, a drive member, a first locking assembly, and a second locking assembly. The housing defines a circular bore. The articulation lever is rotatably supported in relation to the circular bore. The first ratchet has an annular array of teeth supported on the housing. The second ratchet has an annular array of teeth positioned adjacent the first ratchet. The drive member includes a shaft having a first end coupled to the articulation lever. The first locking assembly is supported on the articulation lever and includes a first locking member that is movable from a first position engaged with the first ratchet to a second position disengaged with the first ratchet. The first locking member is configured to prevent rotation of the articulation lever in a counterclockwise direction when the first locking member is in its first position. The second locking assembly is supported on the articulation lever and includes a second locking member that is movable from a first position engaged with the second ratchet to a second position disengaged with the second ratchet. The second locking member is configured to prevent rotation of the articulation lever in a clockwise direction when the second locking member is in its first position.

Other features of the disclosure will be appreciated from the following description.

### BRIEF DESCRIPTION OF DRAWINGS

Various aspects of this disclosure are described herein below with reference to the drawings, wherein:

FIG. 1 is a side perspective view of a surgical stapling device according to aspects of the disclosure with the surgical stapling device in a non-articulated, clamped position;

FIG. 2 is an enlarged view of the indicated area of detail shown in FIG. 1 showing an articulation assembly of the surgical stapling device with a lever of the articulation assembly removed;

FIG. 3 is an exploded perspective view of the articulation assembly shown in FIG. 2;

FIG. 3A is a cross-sectional view taken through a first body half-section of a rotation knob of the surgical stapling device shown in FIG. 1;

FIG. 3B is a perspective view from below of an articulation lever of the articulation assembly shown in FIG. 3;

FIG. 4 is a side perspective view of a first locking assembly of the articulation assembly shown in FIG. 3;

FIG. 5 is a side perspective view of a second locking assembly of the articulation assembly shown in FIG. 3;

FIG. 6 is a cross-sectional view taken through the first locking assembly of the articulation assembly shown in FIG. 2 with the articulation lever shown in phantom;

FIG. 7 is a cross-sectional view taken through the second locking assembly of the articulation assembly shown in FIG. 2 with the lever shown in phantom;

FIG. 8 is a cross-sectional view taken along section line 8-8 of FIG. 6;

FIG. 9 is a cross-sectional view taken along section line 9-9 of FIG. 6;

FIG. 10 is a cross-sectional view taken along section line 10-10 of FIG. 6;

FIG. 11 is a top view of the articulation assembly shown in FIG. 2 with the first locking assembly actuated and in an unlocked position; and

FIG. 12 is a perspective view from above of the surgical stapling device shown in FIG. 1 in an articulated position.

### DETAILED DESCRIPTION

The disclosed surgical device including exemplary aspects of the disclosed articulation assembly will now be described in detail with reference to the drawings in which like reference numerals designate identical or corresponding elements in each of the several views. However, it is to be understood that the aspects of the disclosure included herein are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary manner, while the term “distal” is used generally to refer to that portion of the device that is farther from the clinician during use of the device in its customary manner. In addition, the term “clinician” is used generally to refer to medical personnel including doctors, nurses, surgeons, and support personnel. Further, directional terms such as front, rear, upper, lower, top, bottom, and similar terms are used to assist in understanding the description and are not intended to limit the disclosure.

This disclosure is directed to a surgical device including an elongate body, a tool assembly supported on the elongate body for articulation, and an articulation assembly for selectively articulating the tool assembly in relation to the elongate body. The articulation assembly includes an articulation lever and a pair of locking assemblies. The articulation lever is movable to articulate the tool assembly in relation to the elongate body about an articulation axis. The locking assemblies prevent movement of the articulation lever until one of the locking assemblies is moved from a locked position to an unlocked position. The locking assemblies are movable from their locked positions to their unlocked positions prior to movement of the articulation lever to reduce the force required to manipulate the articulation lever and to facilitate articulation of the tool assembly in continuous fashion.

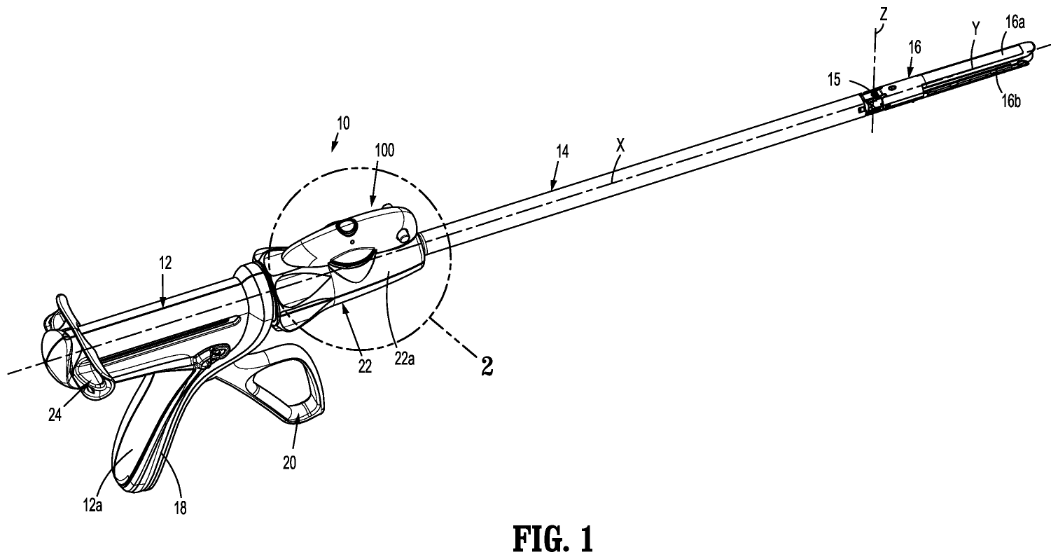

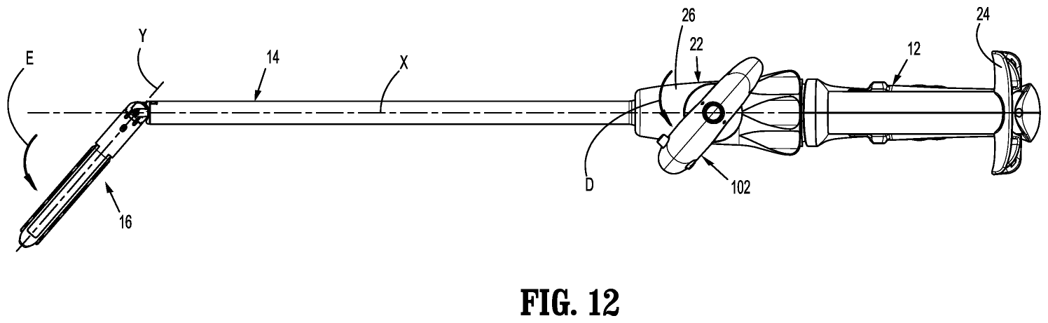

FIG. 1 illustrates a surgical device shown in the form of a stapling device 10 that includes a handle assembly 12, an elongate body or adapter assembly 14, and a tool assembly 16. As illustrated, the handle assembly 12 is manually actuated and includes a stationary handgrip 18, a firing trigger 20, a rotation knob 22, and a retraction knob 24. The handle assembly 12 supports the elongate body 14 and includes a body 12a that defines the stationary handgrip 18. The firing trigger 20 is movable in relation to the stationary handgrip 18 to actuate various functions of the tool assembly 16 via the elongate body 14 including approximation, stapling, and cutting. The handle assembly 12 supports the rotation knob 22 in a manner to facilitate rotation of the rotation knob 22 in relation to the handle assembly 12. The rotation knob 22 supports the elongate body 14 such that rotation of the rotation knob 22 in relation to the handle assembly 12 rotates the elongate body 14 and the tool assembly 16 in relation to the handle assembly 12 about a longitudinal axis “X” defined by the elongate body 14. The rotation knob 22 includes a body 23 that supports an articulation assembly 100 including an articulation lever 102 that is rotatable to articulate the tool assembly 16 as described in further detail below. The tool assembly 16 defines a longitudinal axis “Y-Y” (FIG. 26). The tool assembly 16 is pivotally coupled to the elongate body 14 about a pivot member 15 that defines an articulation axis “Z” to articulate between a non-articulated position in which the longitudinal axes “X” and “Y” of the elongate body 14 and tool assembly 16 are aligned with each other (FIG. 1) and articulated positions in which the longitudinal axes “X” and “Y” of the elongate body 14 and tool assembly 16 are offset from each other (FIG. 12).

The tool assembly 16 is illustrated to be a stapling device and includes an anvil 16a and a cartridge assembly 16b. It is envisioned that the articulation assembly 100 according to aspects of this disclosure could be incorporated into a variety of different endoscopic surgical devices including but not limited to clip appliers, ligation devices, vessel sealing devices, and graspers.

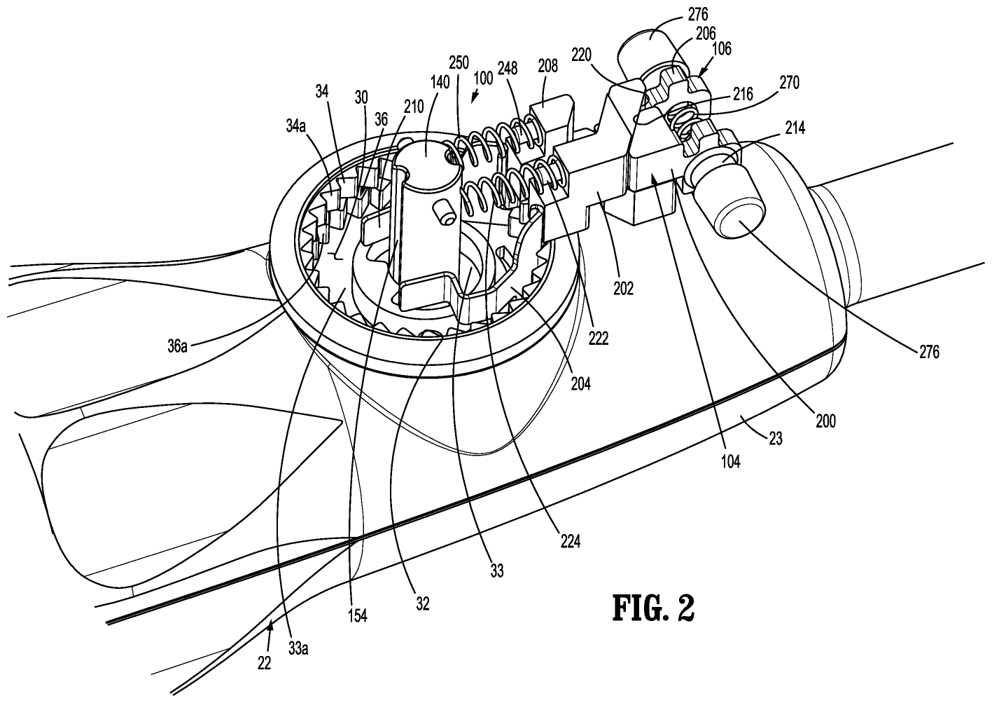

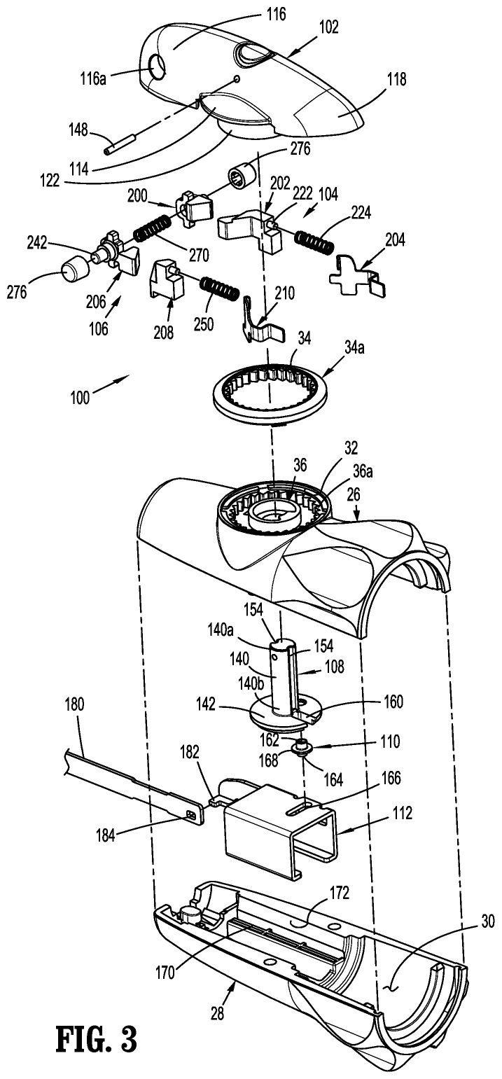

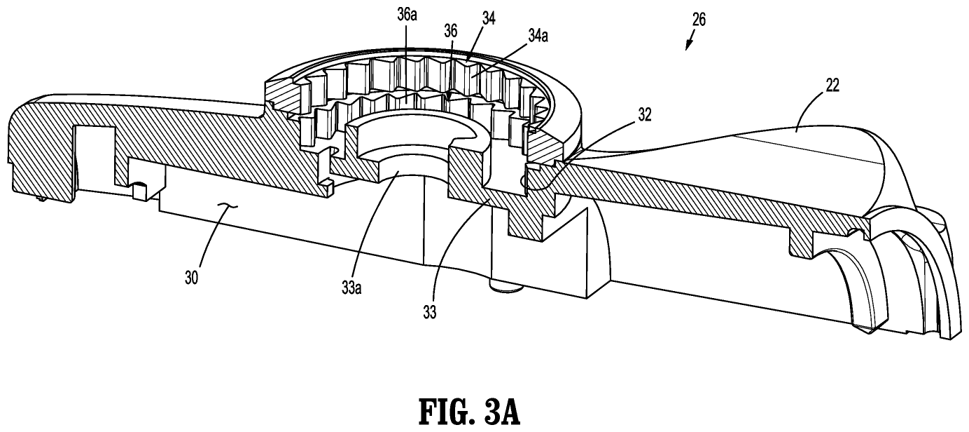

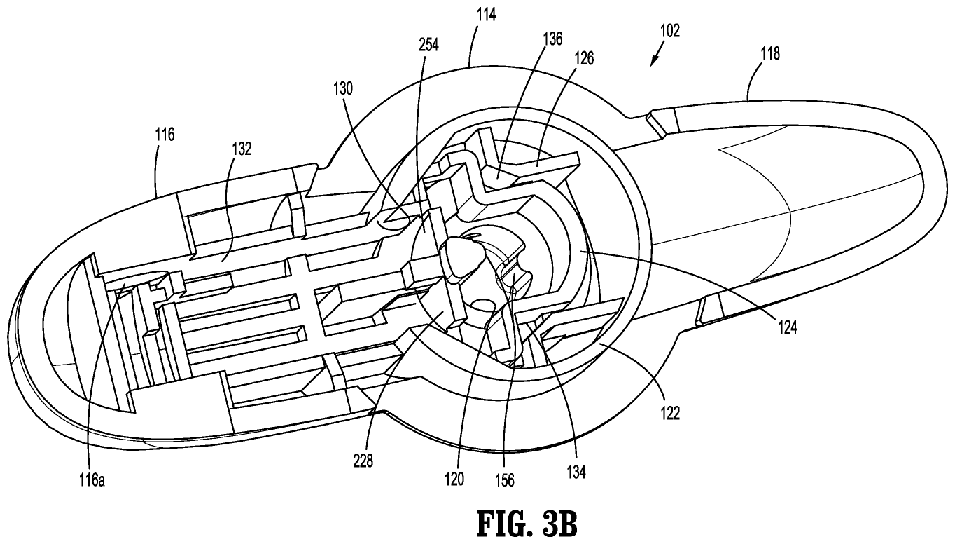

FIGS. 2-3B illustrate the articulation assembly 100 of the stapling device 10 (FIG. 1) which is supported on and within the body 23 of the rotation knob 22. It is envisioned that the articulation assembly 100 need not be supported on the rotation knob but could be supported on a stationary housing of the stapling device 10. In aspects of the disclosure, the body 23 of the rotation knob 22 includes first and second body half-sections 26 and 28 that are secured together to define a cavity 30. The first body half-section 26 defines a circular bore 32 that receives the articulation assembly 100. The circular bore 32 is separated from the cavity 30 by a base wall 33 (FIG. 3A) that defines an opening 33a that extends between the circular bore 32 and the cavity 30. An upper end of the first body half-section 26 defining the circular bore 32 includes or supports a first ratchet 34 and a second ratchet 36. The first ratchet 34 has an annular array of ratchet teeth 34a and the second ratchet 36 has an annular array of ratchet teeth 36a. It is envisioned that the first and second ratchets 34 and 36 could be formed as a single ratchet member. In aspects of the disclosure, the second ratchet 36 has a circular configuration and is integrally formed with the first body half-section 26 and the first ratchet 34 has a circular configuration and is secured to the first body half-section 26 using known fastening techniques or devices, e.g., adhesives, welding, snap-connectors, or combinations thereof. It is envisioned that one or both first and second ratchets 34 and 36 can be integrally formed with the first body half-section 26 or formed separately from and secured to the first body half-section 26. As described in further detail below, the ratchet teeth 34a of the first ratchet 34 are angled in a direction to allow operation of the articulation assembly 100 only in a first direction, e.g., clockwise direction, and the ratchet teeth 36a of the second ratchet 36 are angled to allow operation of the articulation assembly 100 only in a second direction, e.g., counterclockwise direction.

The articulation assembly 100 includes the articulation lever 102, first and second locking assemblies 104 and 106, respectively, a drive member 108, a cam member 110, and a coupling member 112. The articulation lever 102 includes a central hub portion 114, a first extension 116, and a second extension 118. In aspects of the disclosure, the first and second extensions 116 and 118, respectively, are axially aligned with each other and extend outwardly from the central hub portion 114. In certain aspects of the disclosure, the first extension 116 extends distally from the central hub portion 114 and the second extension 118 extends proximally from the central hub portion 114. The first extension 116 defines a transverse through bore 116a. The central hub portion 114 of the articulation lever 102 defines a through bore 120 and includes an outer cylindrical wall 122, an inner retention wall 124, and outer retention walls 126 and 128. The outer cylindrical wall 122 is received within the circular bore 32 of the first body half-section 26 of the rotation knob 22 and defines a cutout 130 that is aligned with a channel 132 defined in the first extension 116 of the articulation lever 102. The channel 132 defined by the first extension 116 communicates with the transverse through bore 116a in the articulation lever 102.

The outer retention walls 126 and 128 are positioned on opposite sides of the inner guide wall 124. In aspects of the disclosure, the outer retention wall 126 and the inner retention wall 124 define a first stepped retention channel 134 on one side of the outer retention wall 124, and the inner retention wall 128 and the outer retention wall 124 define a second stepped retention channel 136 on the other side of the outer retention wall 124. The retention channels 134 and 136 are configured to help to secure the first and second locking assemblies 104 and 106 to the articulation lever 102 as described in further detail below. Other retention channel configurations are envisioned.

The drive member 108 of the articulation assembly 100 includes a central shaft 140 and a disc-shaped member 142. The central shaft 140 extends outwardly of the circular bore 32 in the rotation knob 22 and includes a first end portion 140a that is received within the through bore 120 (FIG. 3B) in the central hub portion 114 of the articulation lever 102 and a second end portion 140b that is fixedly secured to the disc-shaped member 142 within the cavity 30 of the rotation knob 22. The central shaft 140 of the drive member 108 extends through the opening 33a in the base wall 33 of the first body half-section 26 of the rotation knob 22 such that rotation of the articulation lever 102 externally of the cavity 30 of the rotation knob 22 causes corresponding rotation of the central shaft 140 and of the disc-shaped member 142 within the cavity 30. In aspects of the disclosure, the first end portion 140a of the central shaft 140 of the drive member 108 is secured to the articulation lever 102 by a pin 148 that extends through openings 150 and 152 formed in the articulation lever 102 and the central shaft 140, respectively. In certain aspects of the disclosure, the central shaft 140 of the drive member 108 defines elongate grooves 154 that receive protrusions 156 (FIG. 3B) formed within the central hub portion 114 of the articulation lever 102 to rotatably fix the articulation lever 102 to the drive member 108.

The disc-shaped member 142 of the drive member 108 can be integrally formed with the central shaft 140 or secured to the central shaft 140 using known fastening techniques or devices. The disc-shaped member 142 defines a radially extending slot 160 that extends from the central shaft 140 through an outer edge of the disc-shaped member 142.

The cam member 110 is received between the disc-shaped member 142 of the drive member 108 and the coupling member 112 and includes a first camming portion 162 (FIG. 3) and a second camming portion 164. The first camming portion 162 is slidably received within the radially extending slot 160 of the disc-shaped member 142 of the drive member 108 and the second camming portion 164 of the cam member 110 is received in a transverse slot 166 formed in the coupling member 112. In aspects of the disclosure, the camming member 110 includes a central flange 168 that has a diameter that is greater than the width of the transverse slot 166 of the coupling member 112 such that the cam member 110 can slide atop the coupling member 112 along the transverse slot 166 as the drive member 108 is rotated.

In aspects of the disclosure, the coupling member 112 has a rectangular configuration and is supported on shoulders 170 (only one is shown) within a channel 172 (FIG. 3) defined by the second body half-section 28 of the rotation knob 22. The channel 172 is dimensioned to restrict the coupling member 112 to longitudinal movement within the rotation knob 22. When the articulation lever 102 is rotated to rotate the drive member 108, the disc-shaped member 142 of the drive member 108 rotates to move the cam member 110 over a circular path of movement that includes transverse and longitudinal components. As the cam member 110 moves along the circular path of movement, the longitudinal component of movement of the cam member 110 is translated to longitudinal movement of the coupling member 112 within the channel 172 of the rotation knob 22.

The coupling member 112 includes a connector 178 that is coupled to an articulation link 180 (FIG. 3) of the stapling device 10 (FIG. 1). The articulation link 180 includes a distal portion (not shown) that is coupled to the tool assembly 16 such that longitudinal movement of the articulation link 180 causes the tool assembly (FIG. 1) to articulate about the articulation axis “Z” (FIG. 1). In aspects of the disclosure, the connector 178 includes a transversely extending finger 182 that extends through an opening 184 in a proximal portion of the articulation link 180. It is envisioned that a variety of different connector configurations can be used to couple the coupling member 112 to the articulation link 180. For a more detailed description of the construction and operation of a suitable articulation link, see U.S. Pat. No. 7,891,534.

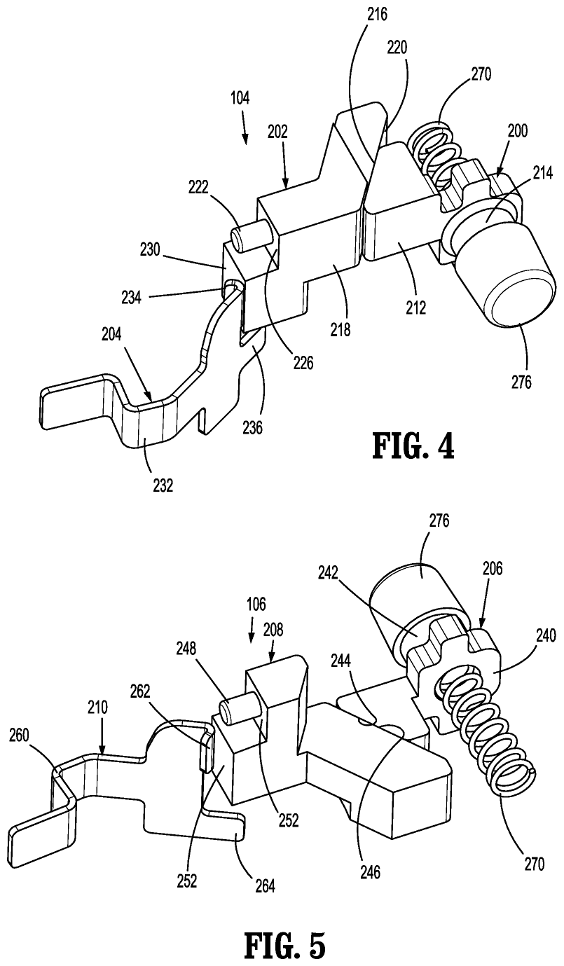

FIGS. 3-7 illustrate the first and second locking assemblies 104 and 106 which are supported within the channel 132 of the articulation lever 102 and extend into circular bore 32 (FIG. 2) defined by the first body half-section 26 of the rotation knob 22. The first locking assembly 104 (FIG. 4) includes an actuator 200, a slider 202, and a locking member 204. Similarly, the second locking assembly 106 (FIG. 5) includes an actuator 206, a slider 208, and a locking member 210. The first and second locking assemblies 104 and 106 are substantially similar in construction and operate in substantially the same manner.

The actuator 200 includes a body 212 (FIG. 4) that has a transversely extending post 214 and an angled cam surface 216. The transversely extending post 214 extends through one side of the transverse through bore 116a (FIG. 3) in the articulation lever 102 and can be pressed inwardly from a non-depressed position to a depressed position into the through bore 116a of the articulation lever 102 to move the body 212 of the actuator 200 inwardly into the channel 132 of the articulation lever 102.

The slider 202 is slidably supported within the channel 132 of the articulation lever 102 and includes an angled cam surface 220 that is in abutting relation with the angled cam surface 216 of the actuator 200. The angled cam surfaces 216 and 220 are configured such that when the actuator 200 is pressed inwardly into the channel 132 of the articulation lever 102, the slider 202 is moved longitudinally in a direction towards the circular bore 32 (FIG. 3) of the rotation knob 22 from a first position towards a second position. The slider 202 includes a post 222 that supports a biasing member, e.g., a coil spring 224, that is positioned to urge the slider 202 towards its first position. In aspects of the disclosure, the coil spring 224 is positioned in compression about the post 222 between a surface 226 of the slider 202 (FIG. 4) and an inner wall 228 (FIG. 3B) of the articulation lever 102. The slider 202 also includes an engagement surface 230.

The locking member 204 is formed of a resilient material, e.g., spring steel, and includes a retention portion 232, an abutment portion 234, and a ratchet finger 236. The retention portion 232 of the locking member 204 is secured within the retention channel 134 (FIG. 3B) of the articulation lever 102. In aspects of the disclosure, the retention portion 232 of the locking member 204 has a stepped configuration that corresponds to the configuration of the retention channel 134. When the articulation lever 102 is secured to the central shaft 140 of the drive member 108 and the slider 202 is in its first position, the ratchet finger 236 of the locking member 204 engages a tooth 34a of the first ratchet 34 (FIG. 3) to limit rotation of the articulation lever 102 to one direction, e.g., a clockwise direction. The ratchet finger 236 is formed of a resilient material that is configured to flex or ratchet over the teeth 34a when the articulation lever 102 is rotated in the permitted direction. Rotation of the articulation lever 102 in the opposite direction, e.g., the counterclockwise direction, is prevented by the engagement of the ratchet finger 236 with one of the teeth 34a of the first ratchet 34.

The abutment portion 234 of the locking member 204 is engaged with the engagement surface 230 of the slider 202. When the slider 202 is moved from its first position to its second position by pressing the actuator 200 into the channel 132 (FIG. 3B) of the articulation lever 102, the locking member 204 is deformed to disengage the ratchet finger 236 from the first ratchet 34 and, thus allow rotation of the articulation lever 102 in both directions.

The actuator 206 includes a body 240 (FIG. 4) that has a transversely extending post 242 and an angled cam surface 244. The transversely extending post 242 extends through the other side of the transverse through bore 116a (FIG. 3) in the articulation lever 102 and can be pressed inwardly into the through bore 116a from a non-depressed position to a depressed position to move the body 240 of the actuator 206 inwardly into the channel 132 of the articulation lever 102.

The slider 208 of the second locking assembly 106 is slidably supported within the channel 132 (FIG. 3B) of the articulation lever 102 and includes an angled cam surface 246 that is in abutting relation with the angled cam surface 244 of the actuator 206. The angled cam surfaces 244 and 246 are configured such that when the actuator 206 is pressed inwardly into the channel 132 (FIG. 3B) of the articulation lever 102, the slider 208 is moved longitudinally in a direction towards the circular bore 32 (FIG. 3) of the rotation knob 22 from a first position towards a second position. The slider 208 includes a post 248 that supports a biasing member, e.g., a coil spring 250, that is positioned to urge the slider 208 towards its first position. In aspects of the disclosure, the coil spring 250 is positioned in compression about the post 248 between a surface 252 of the slider 208 (FIG. 5) and an inner wall 254 (FIG. 3B) of the articulation lever 102. The slider 208 also includes an engagement surface 256.

The locking member 210 is formed of a resilient material, e.g., spring steel, and includes a retention portion 260, an abutment portion 262, and a ratchet finger 264. The retention portion 260 of the locking member 210 is secured within the retention channel 136 (FIG. 3B) of the articulation lever 102. In aspects of the disclosure, the retention portion 260 of the locking member 210 has a stepped configuration that corresponds to the configuration of the retention channel 136. When the articulation lever 102 is secured to the central shaft 140 of the drive member 108 and the slider 208 is in its first position, the ratchet finger 264 of the locking member 210 engages a tooth 36a of the second ratchet 36 (FIG. 3) to limit rotation of the articulation lever 102 to one direction, e.g., a counterclockwise direction. The ratchet finger 264 is formed of a resilient material and is configured to flex or ratchet over the teeth 36a when the articulation lever 102 is rotated in the permitted direction. Rotation of the articulation lever 102 in the opposite direction, e.g., the clockwise direction, is prevented by the engagement of the ratchet finger 264 with the second ratchet 36.

The abutment portion 262 of the locking member 210 is engaged with the engagement surface 252 of the slider 208. When the slider 208 is moved from its first position to its second position by pressing the actuator 206 into the channel 132 (FIG. 3B) of the articulation lever 102, the locking member 210 is deformed to disengage the ratchet finger 264 from the second ratchet 36 and, thus allow rotation of the articulation lever 102 in both directions.

In aspects of the disclosure, each of the actuators 200 and 206 of the first and second locking assemblies 104 and 106, respectively, defines a bore 200a (FIG. 3) and 206a (FIG. 5) that receives a biasing member, e.g., a coil spring 270. The coil spring 270 is positioned between the actuators 200 and 206 to urge the actuators 200 and 206 to their non-depressed positions. In certain aspects of the disclosure, the posts 214 and 242 of actuators 200 and 206 include a cover or cap 276. The caps 276 can be formed of a soft material such as rubber to facilitate more comfortable depression of the actuators 200 and 206 by a clinician.

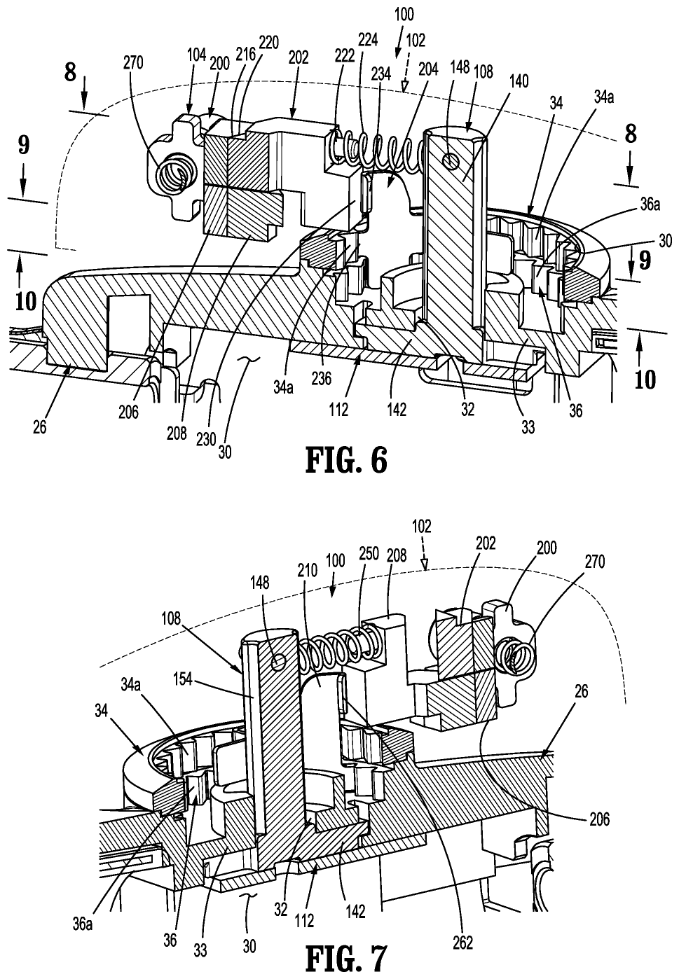

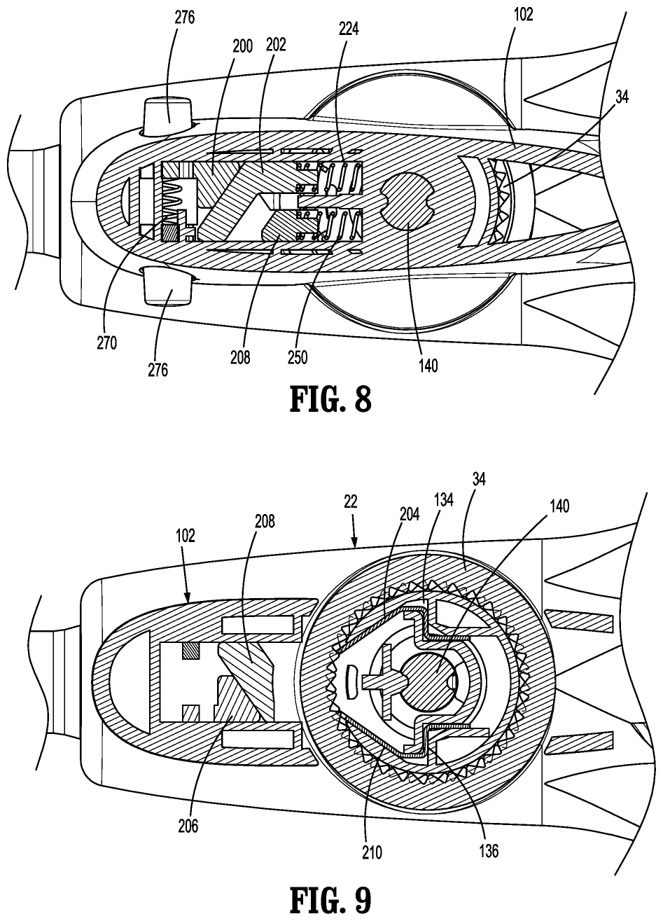

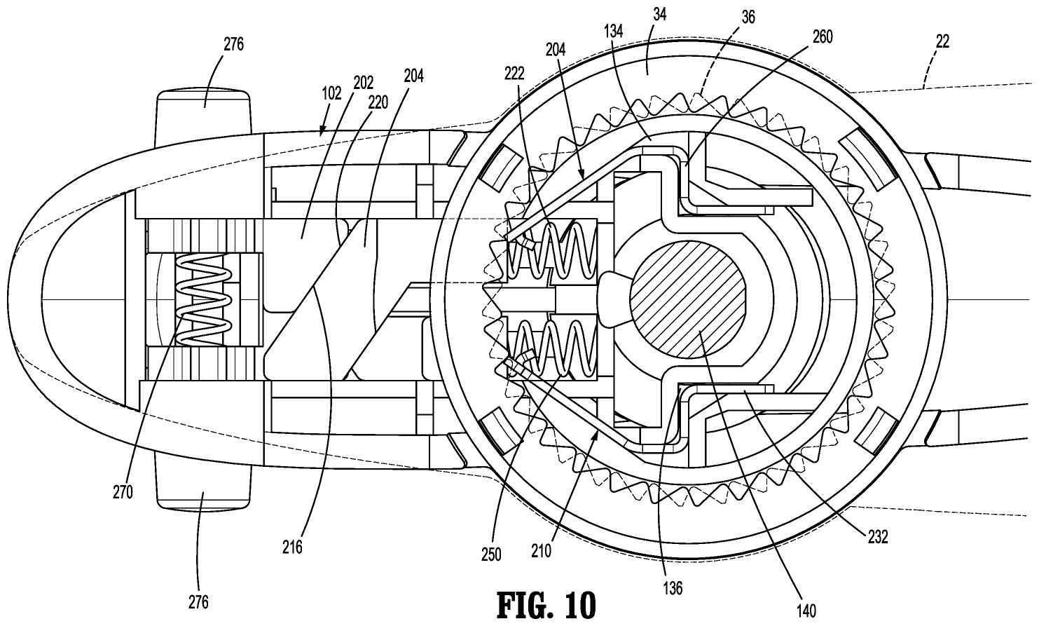

FIGS. 6-10 illustrate the articulation assembly 100 in a locked position. In the locked position, the actuators 200 and 206 of the first and second locking assemblies 104 and 106 are in non-depressed positions and the sliders 202 and 208 are in their first positions. More specifically, the coil spring 270 urges the actuators 200 and 206 of the first and second locking assemblies 104 and 106 to their non-depressed positions, and the coil springs 224 and 250 urge the sliders 202 and 208, respectively, to their first positions. In the first positions of the sliders 202 and 208, the ratchet finger 236 of the locking member 204 is engaged with a tooth 34a of the first ratchet 34 to prevent rotation of the articulation lever 102 in a counterclockwise direction and the ratchet finger 264 of the locking member 210 is engaged with a tooth 36a of the second ratchet 36 to prevent rotation of the articulation lever 102 in a clockwise direction. In this position, the articulation assembly 100 is locked out such that the articulation lever 102 cannot be rotated in either direction.

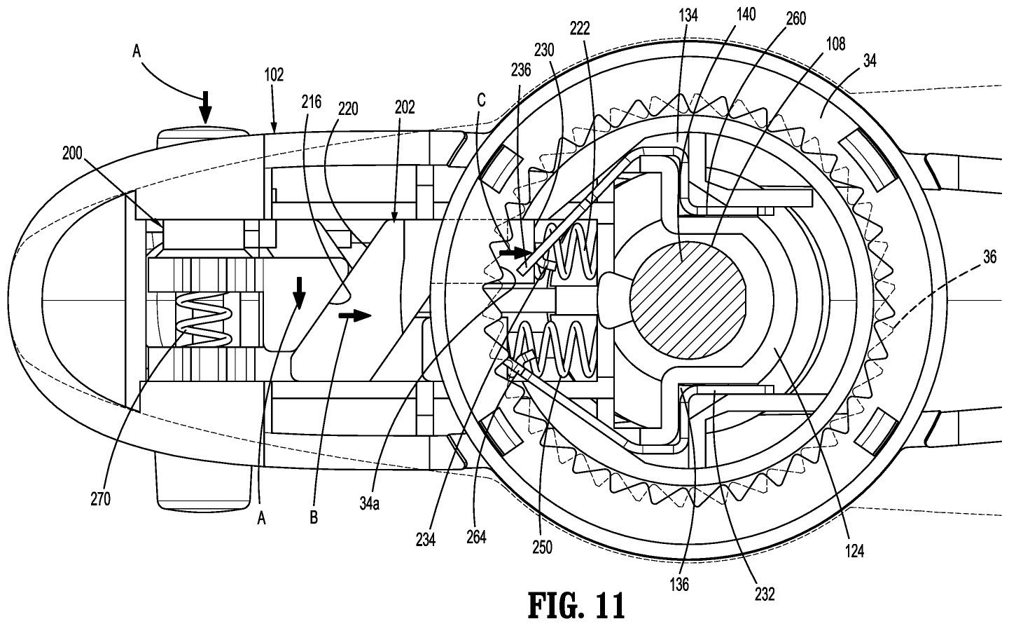

FIGS. 11 and 12 illustrate the articulation assembly 100 when the first locking assembly 104 is unlocked and the articulation lever 102 is rotated in a counterclockwise direction to articulate the tool assembly 16 about the articulation axis “Z” (FIG. 1). When the actuator 200 is depressed in the direction of arrows “A” in FIG. 11 to depress the coil spring 270, the cam surface 216 of the actuator 200 engages the cam surface 220 of the slider 202 to move the slider 202 in the direction of arrow “B” in FIG. 11. When the slider 202 moves in the direction of arrow “B”, the engagement surface 230 on the slider 202 engages the abutment portion 234 of the locking member 204 to deform the locking member 204 inwardly in the direction of arrow “C” to disengage the ratchet finger 236 from the tooth 34a of the first ratchet 34. Once the ratchet finger 236 is disengaged from the first ratchet 34, the articulation lever 102 of the articulation assembly 100 can be rotated in a counterclockwise direction as indicated by arrow “D” in FIG. 12 to articulate the tool assembly 16 in the direction of arrow “E” in FIG. 12 about the articulation axis “Z” (FIG. 1). Operation of the second locking assembly 106 to articulate the tool assembly 16 in the clockwise direction is identical to operation of the first locking assembly 104 described above and will not be described in further detail herein.

It is noted that when the actuator 200 is depressed to unlock the first locking assembly 104, the second locking assembly 106 remains in a locked position to prevent movement of the articulation lever 102 in a clockwise direction. It is envisioned that both actuators 200 and 206 could be simultaneously depressed to simultaneously unlock the first and second locking assemblies 104 and 106 to facilitate selective movement of the articulation lever 102 in either direction.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect of the disclosure may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. An articulation assembly for use with a surgical device comprising:
a housing defining a circular bore and a cavity and including a base wall separating the circular bore from the cavity, the base wall defining an opening that communicates the circular bore with the cavity;
an articulation lever rotatably supported in relation to the circular bore;
a first ratchet having an annular array of teeth supported within the circular bore;
a second ratchet having an annular array of teeth positioned adjacent the first ratchet;
a drive member including a shaft having a first end coupled to the articulation lever and a second end, the shaft extending through the opening in the base wall of the housing, and the drive member rotatable in response to rotation of the articulation lever;
an articulation link coupled to the second end of the drive member such that rotation of the drive member causes longitudinal movement of the articulation link;
a first locking assembly supported on the articulation lever, the first locking assembly including a first locking member that is movable from a first position engaged with the first ratchet to a second position disengaged with the first ratchet, the first locking member is configured to prevent rotation of the articulation lever in a counterclockwise direction when the first locking member is in its first position; and
a second locking assembly supported on the articulation lever, the second locking assembly including a second locking member that is movable from a first position engaged with the second ratchet to a second position disengaged with the second ratchet, the second locking member is configured to prevent rotation of the articulation lever in a clockwise direction when the second locking member is in its first position.

2. The articulation assembly of claim 1, wherein the first locking assembly includes a first actuator that is supported on the articulation lever, and the second locking assembly includes a second actuator that is supported on the articulation lever, the first actuator movable to move the first locking member from its first position to its second position and the second actuator movable to move the second locking member from its first position to its second position.

3. The articulation assembly of claim 2, wherein the first locking assembly includes a first slider that is positioned between the first actuator and the first locking member, and the second locking assembly includes a second slider that is positioned between the second actuator and the second locking member, the first actuator movable from a first position to a second position to move the first slider from a first position to a second position into engagement with the first locking member to move the first locking member from its first to its second position, and the second actuator movable from a first position to a second position to move the second slider from a first position to a second position into engagement with the second locking member to move the second locking member from its first position to its second position.

4. The articulation assembly of claim 3, wherein the first actuator and the first slider include abutting cam surfaces such that transverse movement of the first actuator causes longitudinal movement of the first slider, and wherein the second actuator and the second slider include abutting cam surfaces such that transverse movement of the second actuator causes longitudinal movement of the second slider.

5. The articulation assembly of claim 4, further including a biasing member positioned to urge the first and second actuators towards their first positions.

6. The articulation assembly of claim 4, wherein the first and second locking members are formed of a resilient material, the first locking member including a first retention portion and a first ratchet finger, and the second locking member including a second retention portion and a second ratchet finger, the first ratchet finger engaged with the first ratchet when the first locking member is in its first position and the second ratchet finger engaged with the second ratchet when the second locking member is in its first position.

7. The articulation assembly of claim 6, further including a first biasing member positioned to urge the first slider to its first position and a second biasing member positioned to urge the second slider to its first position.

8. The articulation assembly of claim 1, wherein the second end of the drive member includes a disc-shaped member, and the articulation assembly further includes a cam member and a coupling member, the cam member supported on the disc-shaped member and coupled to the coupling member such that rotational movement of the drive member causes longitudinal movement of the coupling member.

9. The articulation assembly of claim 8, wherein the coupling member is coupled to the articulation link such that longitudinal movement of the coupling member causes longitudinal movement of the articulation link.

10. A surgical device comprising:
an elongate body defining a first longitudinal axis and having a proximal portion and a distal portion;
a tool assembly defining a second longitudinal axis, the tool assembly pivotably supported on the distal portion of the elongate body by a pivot member, the pivot member defining an articulation axis that is transverse to the longitudinal axis, the tool assembly being pivotable about the articulation axis in relation to the elongate body between an articulated position in which the first and second longitudinal axes are coaxial and articulated positions in which the first and second longitudinal axes are misaligned; and
an articulation assembly including:
a housing defining a circular bore and a cavity and including a base wall separating the circular bore from the cavity, the base wall defining an opening that communicates the circular bore with the cavity;
an articulation lever rotatably supported in relation to the circular bore;
a first ratchet having an annular array of teeth supported within the circular bore;
a second ratchet having an annular array of teeth supported adjacent the first ratchet;
a drive member including a shaft having a first end coupled to the articulation lever and a second end, the shaft extending through the opening in the base wall of the housing, and the drive member rotatable in response to rotation of the articulation lever;
an articulation link having a proximal portion and a distal portion, the proximal portion coupled to the second end of the drive member such that rotation of the drive member causes longitudinal movement of the articulation link, the distal portion of the articulation link coupled to the tool assembly such that longitudinal movement of the articulation link causes the tool assembly to move between the non-articulated and articulated positions;
a first locking assembly supported on the articulation lever, the first locking assembly including a first locking member that is movable from a first position engaged with the first ratchet to a second position disengaged with the first ratchet, the first locking member is configured to prevent rotation of the articulation lever in a counterclockwise direction when the first locking member is in its first position; and
a second locking assembly supported on the articulation lever, the second locking assembly including a second locking member that is movable from a first position engaged with the second ratchet to a second position disengaged with the second ratchet, the second locking member is configured to prevent rotation of the articulation lever in a clockwise direction when the second locking member is in its first position.

11. The surgical device of claim 10, wherein the first locking assembly includes a first actuator that is supported on the articulation lever, and the second locking assembly includes a second actuator that is supported on the articulation lever, the first actuator movable to move the first locking member from its first position to its second position and the second actuator movable to move the second locking member from its first position to its second position.

12. The surgical device of claim 11, wherein the first locking assembly includes a first slider that is positioned between the first actuator and the first locking member, and the second locking assembly includes a second slider that is positioned between the second actuator and the second locking member, the first actuator movable from a first position to a second position to move the first slider from a first position to a second position into engagement with the first locking member to move the first locking member from its first to its second position, and the second actuator movable from a first position to a second position to move the second slider from a first position to a second position into engagement with the second locking member to move the second locking member from its first to its second position.

13. The surgical device of claim 12, wherein the first actuator and the first slider include abutting cam surfaces such that transverse movement of the first actuator causes longitudinal movement of the first slider, and wherein the second actuator and the second slider include abutting cam surfaces such that transverse movement of the second actuator causes longitudinal movement of the second slider.

14. The surgical device of claim 13, further including a biasing member positioned to urge the first and second actuators towards their first positions.

15. The surgical device of claim 13, wherein the first and second locking members are formed of a resilient material, the first locking member including a first retention portion and a first ratchet finger, and the second locking member including a second retention portion and a second ratchet finger, the first ratchet finger engaged with the first ratchet when the first locking member is in its first position and the second ratchet finger engaged with the second ratchet when the second locking member is in its first position.

16. The surgical device of claim 15, wherein further including a first biasing member positioned to urge the first slider to its first position and a second biasing member positioned to urge the second slider to its first position.

17. The surgical device of claim 10, wherein the second end of the drive member includes a disc-shaped member, and the articulation assembly further includes a cam member and a coupling member, the cam member supported on the disc-shaped member and coupled to the coupling member such that rotational movement of the drive member causes longitudinal movement of the coupling member.

18. The surgical device of claim 17, wherein the coupling member is coupled to the proximal portion of the articulation link such that longitudinal movement of the coupling member causes longitudinal movement of the articulation link.

19. The surgical device of claim 10, wherein the tool assembly includes an anvil and a cartridge assembly.

20. An articulation assembly for use with a surgical device comprising:
a housing defining a circular bore;
an articulation lever rotatably supported in relation to the circular bore;
a first ratchet having an annular array of teeth supported on the housing;
a second ratchet having an annular array of teeth positioned adjacent the first ratchet;
a drive member including a shaft having a first end coupled to the articulation lever;
a first locking assembly supported on the articulation lever, the first locking assembly including a first locking member that is movable from a first position engaged with the first ratchet to a second position disengaged with the first ratchet, the first locking member is configured to prevent rotation of the articulation lever in a counterclockwise direction when the first locking member is in its first position; and
a second locking assembly supported on the articulation lever, the second locking assembly including a second locking member that is movable from a first position engaged with the second ratchet to a second position disengaged with the second ratchet, the second locking member is configured to prevent rotation of the articulation lever in a clockwise direction when the second locking member is in its first position.
